(19) United States
(12) Patent Application Publication
(10) Pub. No.: US 2024/0041712 A1
(43) Pub. Date: Feb. 8, 2024

(54) COMPOSITION IN THE FORM OF A STABLE MACROSCOPIC EMULSION COMPRISING A PERCENTAGE OF INGREDIENTS OF NATURAL ORIGIN THAT IS GREATER THAN OR EQUAL TO 95% ACCORDING TO ISO STANDARD 16128

(71) Applicant: CAPSUM, MARSEILLE (FR)

(72) Inventor: Sébastien BARDON, PARIS (FR)

(21) Appl. No.: 18/264,210

(22) PCT Filed: Feb. 4, 2022
(86) PCT No.: PCT/EP2022/052678

§ 371 Date: Aug. 3, 2023

(30) Foreign Application Priority Data
Feb. 4, 2021 (FR) 21 01087

(51) Int. Cl. A61K 8/06; A61Q 19/00; A61K 8/92

(52) U.S. Cl. CPC A61K 8/06; A61Q 19/00; A61K 8/922; A61K 2800/34

(57) ABSTRACT

Disclosed is a stable composition, particularly a cosmetic composition, in the form of an emulsion comprising at least one fatty phase and at least one non-miscible aqueous phase at room temperature and atmospheric pressure and in which the dispersed phase is in the form of drops. The drops of dispersed phase that have a diameter greater than or equal to 250 μm represent a volume greater than or equal to 60% of the total volume of the dispersed phase and/or at least 60% of the drops have an average diameter greater than or equal to 250 μm, the composition comprises from 1% to 60% by weight of dispersed phase relative to the total weight of the composition, the composition comprises a percentage of ingredients of natural origin that is greater than or equal to 95% according to ISO standard 16128, and the composition is devoid of amodimethicone.

The object of the present invention pertains to compositions that are in the form of a stable macroscopic emulsion and are comprised of a percentage of ingredients of natural origin that is greater than or equal to 95% according to the standard ISO 16128.

There currently exist stable macroscopic dispersions of droplets comprising a fatty phase dispersed in an aqueous continuous phase, in particular as described in the patent application WO2017046305. These dispersions are generally obtained by making use of a microfluidic manufacturing method and the kinetic stability thereof is ensured thanks to the presence of a membrane (or shell) that is derived from an interfacial complex coacervation reaction which is based in particular on a lipophilic cationic silicone polymer, amodimethicone.

However, there is notably an increasingly strong demand from consumers for cosmetic compositions that are free of silicone compounds on account of the suspected health risks, and/or the environmental impact thereof, given that they are not biodegradable. It would also appear to be desirable to dispense with amodimethicone in the manufacture of such macroscopic dispersions, given the problems relating to compatibility with other raw materials and/or the phenomena of aggregation of the droplets with each other, adhesion of the droplets to the packaging and/or deficiencies in the spherical properties of the droplets, which for obvious reasons would not be desirable. And what's more, the presence of amodimethicone requires the use of suitable solvents, which are generally inorganic or mineral in nature and therefore have natural indexes that are very low, or indeed even zero.

At the same time, it is clear that there is a growing demand among consumers for natural cosmetic compositions. In this context, the standard ISO 16128 has been developed with a view to providing guidelines relating to the definitions applicable to natural and organic cosmetic ingredients at the international level. This standard, published in two parts between 2015 and 2017, sets out the principles and criteria for natural and organic cosmetics and provides a calculation model in order to establish the percentage of ingredients of natural origin, and therefore the naturalness percentage, of a cosmetic product. This calculation model is detailed in Example 1 here below. The Autorité de Régulation Professionnelle de la Publicité (ARPP)/French Professional Advertising Regulatory Authority, indicates that a cosmetic product may only be classified in its entirety as “natural”/“of natural origin” if its natural content/content of natural origin, within the meaning of the standard ISO 16128, is greater than or equal to 95%.

There is therefore a need for new compositions in the form of emulsions comprising droplets of dispersed phase which are macroscopic in size and are endowed with satisfactory properties in terms of kinetic stability and sensory properties upon application, which:
(1) are free of amodimethicone;
(2) allow for the use of high content levels, in particular greater than or equal to 50%, preferably greater than or equal to 75%, and more preferably greater than or equal to 90% by weight relative to the weight of the fatty phase, of oily solvents having natural indexes greater than 0.5; and preferably the use of fatty phases comprising a percentage of ingredients of natural origin that is greater than or equal to 95%, preferably greater than or equal to 96%, in particular greater than or equal to 97%, and better still greater than or equal to 98%, and very particularly greater than or equal to 99%; and
(3) contain in their totality a percentage of ingredients of natural origin that is greater than or equal to 95% (according to the standard ISO 16128).

However, it is known that formulating a conventional stable emulsion is not in and of itself easy, and that phenomena related to instability and/or problems of compatibility are exacerbated with the increase in size of the dispersed phase droplets. Such disadvantages appear to become even more exacerbated given the absence of amodimethicone, and therefore of a membrane, the role of which is precisely to ensure the mechanical resistance of the droplets and the kinetic stability of the emulsion.

It should be noted that the desire to achieve a natural emulsion, that is to say one comprising a percentage of ingredients of natural origin that is greater than or equal to 95% (according to the standard ISO 16128), adds complexity to the problem, since this limits the choice of raw materials and may require making adjustments or adaptations, and/or substitutions to the formulation and/or the manufacturing process. These adaptations and/or substitutions may have an impact, in particular, on the viscosity of the phases, the conductivity of the phases, and/or the surface tensions, which may have consequences that are not negligible, for the proper formation of the emulsion, which is even more so when the emulsion is manufactured based on a microfluidic method. These disadvantages are further exacerbated when it is sought to obtain macroscopic emulsions, which moreover are provided with high levels of fatty phase content.

Thus, the present invention relates to a composition, in particular a cosmetic composition, which is stable in the form of an emulsion comprising at least one fatty phase and at least one aqueous phase which are immiscible with each other at ambient temperature and atmospheric pressure and in which the dispersed phase is in the form of droplets, characterised in that:
the droplets of dispersed phase that have a diameter greater than or equal to 250 μm represent a volume greater than or equal to 60%, or even greater than or equal to 70%, preferably greater than or equal to 80%, and better still greater than or equal to 90% of the total volume of the dispersed phase; and/or at least 60%, or even at least 70%, preferably at least 80%, and better still at least 90%, of the droplets have an average diameter greater than or equal to 250 μm;
the composition comprises from 1% to 60%, in particular from 5% to 50%, preferably from 10% to 40%, and better still from 15% to 30%, by weight of dispersed phase relative to the total weight of the composition;
the composition comprises a percentage of ingredients of natural origin that is greater than or equal to 95%, preferably greater than or equal to 96%, in particular greater than or equal to 97%, and better still greater than or equal to 98%, according to the standard ISO 16128; and
the composition is free of amodimethicone.

In other words, a composition according to the invention is free of, or comprises less than 5%, preferably less than 4%, in particular less than 3%, better still less than 1%, and very particularly less than 0.5% by weight of ingredients that are not of natural origin according to the standard ISO 16128.

Preferably, the fatty phase of a composition according to the invention comprises a percentage of ingredients of natural origin that is greater than or equal to 95%, preferably greater than or equal to 96%, in particular greater than or equal to 97%, better still greater than or equal to 98%, and most particularly greater than or equal to 99%. This characteristic feature is completely unprecedented, as it cannot be achieved with the fatty phases of the prior art, given that it is necessary to use lipophilic solvents that are capable of satisfactorily solubilising amodimethicone. However, such lipophilic solvents are generally inorganic (or mineral) in nature, and therefore have natural indexes that are very low, or indeed even zero.

As can be seen from the examples here below, the inventors have in an unexpected manner succeeded in obtaining such compositions in the form of stable macroscopic emulsions that offer satisfactory levels of performance in terms of kinetic stability and therefore visual and aesthetic appearance, and ease and comfort of application to the skin, while at the same time achieving percentages of naturalness that are greater than or equal to 95% (according to the standard ISO 16128).

In particular, the inventors have observed that a composition according to the invention exhibits satisfactory performance levels in terms of the droplets not aggregating together, the droplets not adhering to the packaging, and in terms of comfort and ease of application to the skin.

Within the meaning of the present invention, the terms “stable” or “kinetic stability” are understood to indicate, at ambient temperature and atmospheric pressure: the absence of creaming or sedimentation of the dispersed phase droplets in the continuous phase, the absence of opacification of the aqueous phase, the absence of aggregation of the droplets with each other, and in particular the absence of Ostwald ripening or coalescence of the droplets with one another, the absence of adhesion of the droplets to the packaging, and the absence of leakage of matter from the dispersed phase into the continuous phase, or vice versa; for a composition according to the invention, over a period of time that is greater than or equal to 1 month, preferably greater than or equal to 3 months, and better still greater than or equal to 6 months.

In the context of the present invention, the terms “emulsion” or “dispersion” may be used interchangeably without distinction to refer to the abovementioned compositions.

In the context of the present invention, the dispersed phase droplets may be referred to indifferently by the term “droplets (G1)”.

The terms “macroscopic” or “macroscopic droplet” are used within the meaning of the present invention to refer to dispersed phase droplets that are visible to the naked eye, as opposed to microscopic droplets that are not visible to the naked eye. Thus, in a composition according to the invention:
droplets having a diameter greater than or equal to 250 μm, in particular greater than or equal to 500 μm, preferably greater than or equal to 750 μm, or even greater than or equal to 1,000 μm, or indeed even between 250 μm and 3,000 μm, better still between 500 μm and 2000 μm, and in particular between 750 μm and 1000 μm, represent a volume greater than or equal to 60%, or even greater than or equal to 70%, preferably greater than or equal to 80%, and better still greater than or equal to 90%, of the total volume of the dispersed phase; and/or
at least 60%, or even at least 70%, preferably at least 80%, and better still at least 90%, of the droplets have an average diameter greater than or equal to 250 μm, in particular greater than or equal to 500 μm, preferably greater than or equal to 750 μm, or even greater than or equal to 1,000 μm, or indeed even between 250 μm and 3,000 μm, better still between 500 μm and 2,000 μm, and in particular between 750 μm and 1,000 μm.

The determination of the volume of droplets having a particular diameter in relation to the total volume of the dispersed phase falls within the scope of the general knowledge of the person skilled in the art, particularly with regard to the measurement method for measuring the diameter as described here below.

The droplets of dispersed phase are preferably flexible solids. According to the invention, the term “flexible solid” is used to refer in particular to the fact that the droplets do not flow under their own weight, but can be deformed by pressure, for example by applying pressure with a finger. In this way, they have a consistency similar to that of butter (without the fatty nature), and are malleable and easy to grip. The droplets can be spread easily by hand, particularly on keratinous matter, in particular the skin.

According to one other embodiment, a composition according to the invention comprises less than 0.5%, preferably less than 0.25%, and in particular less than 0.05%, by weight of surfactant(s) relative to the total weight of the composition, and preferably does not comprise any surfactant.

According to one embodiment, a composition according to the invention does not contain any of the following: synthetic fragrances; colouring agents; synthetic pigments; ethoxylated raw materials, for example polyethylene glycol (PEG); silicone; mineral oils; nanometric raw materials (or nanomaterials); sodium lauryl sulphate (or SLS); raw materials which have undergone irradiation; butylated hydroxy toluene (BHT); butylated hydroxy anisole (BHA); raw materials derived from genetically modified organisms (GMO); benzophenone; ethylenediaminetetraacetic acid (EDTA); and preservatives selected from parabens, phenoxyethanol, and mixtures thereof.

Preferably, the droplets of dispersed phase of a composition according to the invention advantageously exhibit an apparent monodispersity (that is to say, they are perceived by the naked eye as being spheres that are identical in diameter). The droplets advantageously are substantially spherical.

According to a first embodiment, the droplets of a composition according to the invention are devoid of membrane (or shell), in particular of polymeric membrane or membrane formed by interfacial polymerisation. In particular, the droplets of a composition according to the invention are not stabilised by means of a coacervate membrane (of the type: anionic polymer (carbomer)/cationic polymer (amodimethicone)). In other words, there is direct contact between the continuous aqueous phase and the dispersed fatty phase.

Thus, according to this first embodiment, a composition according to the invention does not comprise a shell, in particular a shell formed by a layer of coacervate interposed between the dispersed fatty phase and the continuous aqueous phase. In particular, a composition according to the invention does not comprise (is devoid of) a lipophilic cationic polymer corresponding to the following formula (I):

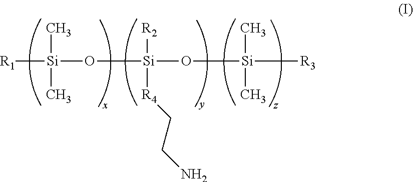

in which:
R₁, R₂ and R₃, independently of each other, represent OH or CH₃;
R₄ represents a —CH₂— group or an —X—NH— group in which X is a divalent alkylene radical having 3 carbon atoms (C3) or 4 carbon atoms (C4);
x is an integer between 10 and 5000, preferably between 30 and 1000, and better still between 80 and 300;
y is an integer between 1 and 1000, in particular between 2 and 1000, preferably between 4 and 100, and better still between 5 and 20; and
z is an integer between 0 and 10, preferably between 0 and 1, and better still is equal to 1.

According to a second variant embodiment, the droplets of a composition according to the invention comprise a membrane (or shell), in particular a polymeric membrane or a membrane formed by interfacial polymerisation. In particular, the droplets of a composition according to the invention are stabilised by means of a coacervate membrane (of the type: anionic polymer (carbomer)/cationic polymer, where the cationic polymer is other than a silicone polymer or a derivative thereof, and in particular other than amodimethicone). In other words, the contact between the continuous aqueous phase and the dispersed fatty phase is not direct.

Thus, according to this second variant embodiment, a composition according to the invention comprises a shell, in particular a shell formed by a layer of coacervate interposed between the dispersed phase and the continuous phase. In particular, a composition according to the invention comprises at least one lipophilic cationic polymer corresponding to formula (I) here above.

Preferably, the droplets of a composition according to the invention differ from solid capsules, that is to say capsules provided with a solid shell (or “membrane”), such as for example those described in the document WO2010/063937.

According to the invention, the pH of a composition is typically between 4.0 and 8.0, in particular between 5.0 and 7.0.

### Temperature and Pressure

Unless otherwise stated, in all the sections that follow, it is assumed that ambient temperature (for example T=25° C.±2° C.) and atmospheric pressure (760 mm Hg, that is 1.013.105 Pa or 1013 mbar) are prevailing.

### Viscosity

The viscosity of a composition according to the invention or of at least one of the phases thereof may vary to a significant degree, which makes it possible to obtain varied textures. The viscosity is measured at ambient temperature and at ambient pressure according to the method described in the document WO2017046305.

According to one embodiment, a composition according to the invention has a viscosity ranging from 1 mPa·s to 500,000 mPa·s, preferably from 10 mPa·s to 300,000 mPa·s, better still from 400 mPa·s to 100 000 mPa·s, and more particularly from 1,000 mPa·s to 30,000 mPa·s, as measured at 25° C. according to the method described here above.

The viscosity of a phase may be increased thanks to the presence of at least one gelling agent. For the purposes of this invention, the term “gelling agent” is used to refer to an agent which makes it possible to increase the viscosity of the phase that is devoid of the said gelling agent, and preferably to achieve a final viscosity of the phase thus gelled that is greater than or equal to 2,000 mPa·s, preferably greater than or equal to 5,000 mPa·s, more preferably greater than or equal to 10,000 mPa·s, and very particularly greater than or equal to 20,000 mPa·s. According to one embodiment, a phase comprising a gelling agent according to the invention has a viscosity ranging from 2,000 mPa·s to 50,000 mPa·s, preferably from 3,000 mPa·s to 30,000 mPa·s, better still from 4,000 mPa·s to 20,000 mPa·s, and more particularly from 5,000 mPa·s to 10,000 mPa·s, as measured at 25° C. according to the method described here above.

### The Standard ISO 16128

The standard ISO 16128 provides guidelines for definitions applicable to natural and organic cosmetic ingredients at the international level. This standard is commonly used for the purpose of defining the naturalness of a product.

ISO 16128 consists of the following two parts:
Part 1: Definitions for ingredients; and
Part 2: Criteria for ingredients and products

This standard defines 3 different categories of ingredients:

### 1—Synthetic Ingredients

This category refers to purely chemical ingredients that have been synthesised in the laboratory. The natural index for ingredients in this category is therefore 0. Included in this category, are to be found many preservatives, synthetic fragrances, and even certain active ingredients.

### 2—Natural Ingredients of Plant, Mineral or Animal Origin

Included in this category, are to be found ingredients derived from nature that have not undergone any chemical transformation. The natural index thereof is therefore 1. For example, the following may be found:
natural ingredients of plant origin such as, for example, virgin plant oils like argan, almond, or wheat oil, etc;
natural ingredients of mineral origin such as for example minerals like zinc or magnesium; and
natural ingredients of animal origin such as, for example, honey.

### 3—Ingredients Referred to as “Naturally Derived” Ingredients

In this category, the ingredients are of natural origin but have undergone chemical transformations, that is to say, their natural state has been modified in order to be able to use them in cosmetics. For these ingredients, it is necessary to calculate their natural index, that is to say, to calculate the fraction of the ingredient that is derived from nature. This calculation is established by the supplier of the raw material.

An ingredient shall be deemed to be “naturally derived” if its natural index is greater than 0.5, that is to say if it contains more than 50% of natural raw materials. If its result happens to be less than 0.5, that is to say containing less than 50% of natural raw materials, the natural index considered is 0.

The calculation of the natural index of a raw material, where necessary in the presence of a solvent, falls within the scope of the general knowledge of the person skilled in the art.

By way of illustration, the calculation of the natural index (NI) of cocamidopropyl betaine, a surfactant derived from natural lauric acid and a synthetic amine, is described here below.

IN=M_{natural fraction}/M_{final molecule}

IN−183 g/mol/343 g/mol=0.533

The calculation of the natural index is generally established and communicated by the supplier of the raw material. For the same given ingredient, having a given chemical structure, it is therefore possible to obtain different natural indexes, depending on the method of preparation used for preparing the said ingredient.

This standard provides a calculation model for use in order to establish the percentage of ingredients of natural origin, and therefore the percentage of naturalness, of a cosmetic product on the basis of the natural indexes of the raw materials that form the composition. This calculation model is detailed in Example 1 here below.

A composition according to the invention comprises a percentage of ingredients of natural origin that is greater than or equal to 95%, preferably greater than or equal to 96%, in particular greater than or equal to 97%, and better still greater than or equal to 98%.

This percentage is assessed in relation to the percentage of the totality of all the ingredients (or raw materials) of the composition.

### Composition

A composition according to the invention may be in the form of a single emulsion, in particular of the type: oil-in-water, or water-in-oil; or in the form of a multiple emulsion, in particular of the type: oil-in-water-in-oil, water-in-oil-in-water, or oil-in-oil-in-water.

According to a first embodiment, a composition according to the invention is a single emulsion, that is to say containing only one aqueous phase and one fatty phase. With regard to the nature of the phases, a composition according to the invention may be an emulsion of the oil- in- water type (or direct emulsion), or an emulsion of the water- in- oil type (or invert emulsion), the fatty phase and the aqueous phase being immiscible with each other at ambient temperature and atmospheric pressure. Thus, the solubility of the fatty phase in the aqueous phase is advantageously less than 5% by mass, and vice versa.

According to one other embodiment, a composition according to the invention is a multiple emulsion, in particular a double emulsion, for example of the type: water-in-oil-in-water, oil-in water-in-oil, or oil-in-oil-in-water.

Various pairs of oils that are not miscible with each other are described in particular in the French patent application FR3063893.

Regardless of the embodiment concerned, a composition according to the invention may be characterised as a macroscopically inhomogeneous mixture of two immiscible phases. In other words, in a composition according to the invention, the continuous phase can be distinguished from the dispersed phase, and vice versa, in particular with the naked eye.

The droplets G1 of dispersed phase may be:
monophasic;
   or
multiphasic, in which the droplets G1 contain an intermediate droplet of an intermediate phase, the intermediate phase being placed in contact with the continuous phase (where applicable separated by a membrane when present), and at least one, preferably one single, internal droplet of an internal phase disposed within the intermediate droplet.

The intermediate phase is preferably made from a solution that is immiscible with the continuous phase, and the intermediate phase is preferably made from a solution that is immiscible with the internal phase, at ambient temperature and atmospheric pressure. Thus, the solubility of the intermediate phase with the continuous phase and the internal phase is advantageously less than 5% by mass, and vice versa, in which case:
when the continuous phase is aqueous, the intermediate phase is oily and the internal phase is aqueous;
when the continuous phase is oily, the intermediate phase is aqueous and the internal phase is oily; and
when the continuous phase is aqueous, the intermediate phase is oily and the internal phase is oily.

Advantageously, the intermediate phase comprises at least one gelling agent, in particular as defined here below. The gelling agent contributes in particular to enhancing the suspension of the one or more internal droplet(s) disposed within the intermediate droplet and prevents/avoids the phenomena of creaming or sedimentation of the one or more internal droplet(s) disposed within the intermediate droplet.

### Aqueous Phase

A composition according to the invention comprises at least one aqueous phase.

The aqueous phase may feature the continuous phase or the dispersed phase of a composition according to the invention, preferably the aqueous phase features the continuous phase of a composition according to the invention.

When the continuous phase of a composition according to the invention is an aqueous phase, the latter is preferably in the form of a gel, in particular a gel having a viscosity that is suitable for suspending the droplets of dispersed phase and thus contributing to the kinetic stability and visual attractiveness of a composition according to the invention.

Advantageously, in particular when it features as the continuous phase, the aqueous phase is not solid at ambient temperature and ambient pressure, that is to say it is capable of flowing under its own weight.

According to one embodiment, the aqueous phase has a viscosity of between 400 mPa·s and 100,000 mPa·s, preferably between 800 mPa·s and 30,000 mPa·s, as measured at 25° C. according to the method as described here above.

An aqueous phase comprises at least water. In addition to distilled or deionised water, a water type that is appropriate for the invention may also be natural spring water or floral water.

According to one embodiment, the percentage by mass of water in the aqueous phase is at least 30%, preferably at least 40%, in particular at least 50%, and better still at least 60%, in particular between 70% and 98%, and preferably between 75% and 95%, relative to the total mass of the said aqueous phase.

The aqueous phase, in particular when it constitutes the continuous phase of a composition according to the invention, may in addition comprise at least one base. It may comprise one single base or a mixture of multiple different bases. The presence of at least one base in such an aqueous continuous phase contributes in particular to increasing the viscosity of the latter. In the remainder of the description, this base may also be referred to as a “viscosity-increasing solution”. In one embodiment, the base present in the aqueous phase is an inorganic (or mineral) base.

According to one embodiment, the inorganic/mineral base is selected from the group constituted of alkali metal hydroxides and alkaline earth metal hydroxides. Preferably, the inorganic/mineral base is an alkali metal hydroxide, and in particular NaOH.

According to one embodiment, the base present in the aqueous phase is an organic base. Among the organic bases, mention may be made, for example, of aqueous ammonia, pyridine, triethanolamine, aminomethylpropanol, arginine, or even triethylamine.

A composition according to the invention may comprise from 0.01% to 10%, preferably from 0.01% to 5%, and preferentially from 0.02% to 1%, by weight of one or more base(s), preferably of inorganic/mineral base(s), and in particular of NaOH, relative to the total weight of the composition, and better still relative to the total weight of the aqueous phase containing the said base(s).

### Fatty Phase

A composition according to the invention comprises at least one fatty phase, also referred to in the remainder of the description as the “oily phase”.

The fatty phase may represent either the continuous phase or the dispersed phase of a composition according to the invention. Preferably, the fatty phase represents the dispersed phase of a composition according to the invention.

Preferably, a fatty phase of a composition according to the invention has a melting point of between 50° C. and 100° C., preferably between 60° C. and 90° C.

The melting point of a fatty phase may be measured by means of a differential scanning calorimeter (DSC), for example the calorimeter sold under the trade name “DSC Q2000” by the company TA Instruments. The protocols for sample preparation and measurement are as follows: a 5 mg sample of the sample to be tested, which has been previously heated to 80° C. and collected under magnetic stirring with the aid of a spatula that has also been heated, is placed in a hermetically sealed aluminium capsule, or crucible. Two tests were carried out in order to ensure reproducibility of the results. The measurements are carried out on the calorimeter mentioned here above. The furnace is subjected to a nitrogen flush. Cooling is then carried out by means of the RCS 90 heat exchanger. The sample is subsequently subjected to the following protocol: it is first heated to 20° C., then subjected to a first increase in temperature from 20° C. to 130° C. at a heating rate of 5° C./minute, then cooled from 130° C. to −80° C. at a cooling rate of 5° C./minute, and finally subjected to a second increase in temperature from −80° C. to 130° C. at a heating rate of 5° C./minute. During the second temperature increase, the variation in the difference in power absorbed by the empty crucible and the crucible containing the sample is measured as a function of temperature. The melting point of the compound is the value of the temperature corresponding to the apex of the peak of the curve representing the variation in the difference in power absorbed as a function of temperature. The end of melting point range corresponds to the temperature at which 95% of the sample has melted.

A fatty phase of a composition according to the invention advantageously comprises at least one lipophilic gelling agent, preferably one that offers a natural index greater than 0.5, in particular greater than or equal to 0.75, better still greater than or equal to 0.85, and most particularly greater than or equal to 0.95 (according to the standard ISO 16128).

### Lipophilic Gelling Agent

Advantageously, a lipophilic gelling agent is a thermo-sensitive gelling agent, that is to say one which reacts to heat, and in particular is a gelling agent which is solid at ambient temperature and liquid at temperatures higher than 50° C., preferably higher than 60° C., and better still higher than 70° C. Preferably, a thermo- sensitive lipophilic gelling agent according to the invention has a melting point of between 50° C. and 130° C., and preferably between 60° C. and 120° C.

The lipophilic gelling agent according to the invention may be selected from among organic or inorganic, polymeric or molecular lipophilic gelling agents; fats that are solid at ambient temperature and pressure; and mixtures thereof.

### Organic or Inorganic, Polymeric or Molecular Lipophilic Gelling Agent(s)

By way of inorganic lipophilic gelling agents, mention may be made of clays that may be modified, for instance hectorites modified with a C₁₀ to C₂₂ ammonium chloride, such as hectorite modified with di-stearyl di-methyl ammonium chloride such as, for example, the product marketed under the trade name Bentone 38VÒ by the company ELEMENTIS. Mention may also be made of hectorite modified with distearyldimethylammonium chloride, also known as quaternium-18 bentonite, such as the products manufactured or marketed under the trade names Bentone 34 by the company Rheox; Claytone XL, Claytone 34, and Claytone 40 manufactured or marketed by the company Southern Clay; modified clays known as benzalkonium and quaternium-18 bentonites and marketed or manufactured under the trade names Claytone HT, Claytone GR and Claytone PS by Southern Clay, clays modified with stearyldimethylbenzoylammonium chloride, also known as steralkonium bentonites, such as the products manufactured or marketed under the trade names Claytone APA and Claytone AF by the company Southern Clay; and Baragel 24 manufactured or marketed by the company Rheox.

The lipophilic, organic, polymer gelling agents are, for example, ethylcellulose such as the product sold under the trade name EthocelÒ by the company DOW CHEMICAL; galactommanans containing from one to six, and in particular from two to four, hydroxyl groups per ose, substituted by a saturated or unsaturated alkyl chain, such as guar gum alkylated by C1 to C6, and in particular C1 to C3 alkyl chains, and mixtures thereof. As well, “diblock”, “triblock” or “radial” type block copolymers of the type polystyrene/polyisoprene, polystyrene/polybutadiene such as those marketed under the trade name Luvitol HSB® by the company BASF; of the type polystyrene/copoly(ethylene- propylene) such as those marketed under the trade name Kraton® by the company SHELL CHEMICAL CO; or even of the type polystyrene/copoly(ethylene-butylene); mixtures of triblock and radial (star) copolymers in isododecane, such as those marketed by the company PENRECO under the trade name Versagel®, for example the mixture of butylene/ethylene/styrene triblock copolymer and ethylene/propylene/styrene star copolymer in isododecane (Versagel M 5960).

According to one embodiment, the gelling agents that may be used according to the invention may be selected from the group constituted of esters of sugar/polysaccharide and fatty acid(s), in particular esters of dextrin and fatty acid(s), esters of glycerol and fatty acid(s), or esters of inulin and fatty acid(s); polyamides, and mixtures thereof.

By way of lipophilic gelling agent, mention may also be made of polymers having a weight-average molecular weight of less than 100,000, which comprise a) a polymer backbone having hydrocarbon repeat units provided with at least one heteroatom; and optionally b) at least one pendant fatty chain and/or at least one optionally functionalised terminal fatty chain, having from 6 to 120 carbon atoms and being bonded to these hydrocarbon units, as described in the patent applications WO 02/056847, WO 02/47619, in particular polyamide resins (in particular comprising alkyl groups having from 12 to 22 carbon atoms) such as those described in the patent application U.S. Pat. No. 5,783,657.

By way of example of a polyamide resin that may be used in accordance with the present invention, mention may be made of UNICLEAR 100 VG® marketed by the company ARIZONA CHEMICAL.

Among the lipophilic gelling agents which can be used in the present invention, mention may also be made of esters of dextrin and fatty acid, such as dextrin palmitates. According to one embodiment, the ester of dextrin and one or more fatty acid(s) according to the invention is a mono- or poly-ester of dextrin and at least one fatty acid corresponding to the following formula (II):

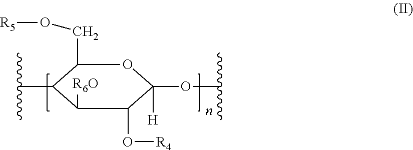

in which:
n is an integer ranging from 2 to 200, preferably ranging from 20 to 150, and in particular ranging from 25 to 50;
the radicals R4, R5 and R6, which are identical or different, are selected from hydrogen or an acyl group -CORa in which the radical Ra represents a hydrocarbon radical, either linear or branched, saturated or unsaturated, having from 5 to 50, preferably from 5 to 25 carbon atoms;
provided that at least one of the said radicals R4, R5 or R6 is other than hydrogen.

Among the esters of dextrin and one or more fatty acid(s), mention may be made, for example, of dextrin palmitates, dextrin myristates, dextrin palmitates/ethylhexanoates and mixtures thereof. Mention may in particular be made of esters of dextrin and one or more fatty acid(s) marketed under the trade names Rheopearl® KL2 or D2 (INCI [International Nomenclature of Cosmetic Ingredients] name: dextrin palmitate), Rheopearl® TT2 (INCI name: dextrin palmitate ethylhexanoate), and Rheopearl® MKL2 (INCI name: dextrin myristate) by the company Miyoshi Europe. Among the lipophilic gelling agents that can be used in the present invention, mention may also be made of the esters of inulin and one or more fatty acid(s) marketed under the trade names Rheopearl® ISK2 or Rheopearl® ISL2 (INCI name: Stearoyl Inulin) by the company Miyoshi Europe.

Among the lipophilic gelling agents that can be used in the present invention, mention may also be made of esters of glycerol and one or more fatty acid(s), in particular a mono-, di- or triester of glycerol and one or more fatty acid(s). Typically, the said ester of glycerol and fatty acid(s) may be used alone or in a mixture. According to the invention, it may be an ester of glycerol and a fatty acid or an ester of glycerol and a mixture of fatty acids. In one embodiment, the fatty acid is selected from the group constituted of behenic acid, isooctadecanoic acid, stearic acid, eicosanoic acid, and mixtures thereof. According to one embodiment, the ester of glycerol and one or more fatty acid(s) has the following formula (III):

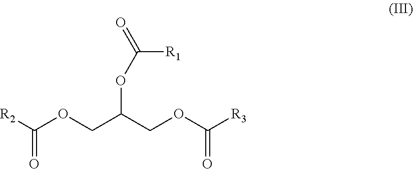

in which: R₁, R₂ and R₃, independently of one another, are selected from H and a saturated alkyl chain comprising from 4 to 30 carbon atoms, at least one of R₁, R₂ and R₃ being other than H. According to one embodiment, R₁, R₂ and R₃ are different. In particular mention may be made of the esters of glycerol and one or more fatty acid(s) marketed under the trade names Nomcort HK-G (INCI name: Glyceryl behenate/eicosadioate) and Nomcort SG (INCI name: Glyceryl tribehenate, isostearate, eicosadioate), by the company Nisshin Oillio.

### Solid Fatty Substances

The fatty substance that is solid at ambient temperature and pressure is selected in particular from the group constituted of waxes, pasty fatty substances, butters and mixtures thereof.

### Wax(es)

For the purposes of the invention, the term “wax” is used to refer to a lipophilic compound, which is solid at ambient temperature (25° C.), with a reversible solid/liquid change of state, and having a melting point that is higher than or equal to 50° C. and can go up to 120° C.

The protocol for measuring this melting point is as previously described.

The waxes that may be used in a dispersion according to the invention may be selected from solid waxes, whether or not deformable at ambient temperature, of animal, plant, mineral or synthetic origin, and mixtures thereof. In particular, use may be made of hydrocarbon waxes such as beeswax, lanolin wax and China insect wax; rice wax, Carnauba wax, Candellila wax, Ouricurry wax, Alfa wax, cork fibre wax, sugar cane wax, Japan wax and sumac wax; montan wax, microcrystalline waxes, paraffins and ozokerite; polyethylene waxes, waxes obtained by means of the Fisher-Tropsch synthesis, and waxy copolymers and the esters thereof. Mention may be made in particular of waxes marketed under the trade names Kahlwax®2039 (INCI name: Candelilla cera) and Kahlwax®6607 (INCI name: Helianthus Annuus Seed Wax) by the company Kahl Wachsraffinerie, Casid HSA (INCI name: Hydroxystearic Acid) by the company SACI CFPA, Performa®260 (INCI name: Synthetic wax), and Performa®103 (INCI name: Synthetic wax) by the company New Phase, and AJK-CE2046 (INCI name: Cetearyl alcohol, dibutyl lauroyl glutamide, dibutyl ethylhaxanoyl glutamide) by the company Kokyu Alcohol Kogyo. Mention may also be made of waxes obtained by catalytic hydrogenation of animal or plant oils containing linear or branched, C8-C32 fatty chains. Among the latter, in particular mention may be made of hydrogenated jojoba oil, hydrogenated sunflower oil, hydrogenated castor oil, hydrogenated coconut oil, and hydrogenated lanolin oil, di-(trimethylol -1,1,1-propane) tetrastearate sold under the trade name “HEST 2T-4S” by the company HETERENE, di-(trimethylol -1,1,1-propane) tetrabenate sold under the trade name HEST 2T-4B by the company HETERENE.

It is also possible to use the waxes obtained by transesterification and hydrogenation of plant oils, such as castor oil or olive oil, for instance the waxes sold under the trade names Phytowax Ricin 16L64® and 22L73® and Phytowax Olive 18L57 by the company SOPHIM. Such waxes are described in the French patent application FR2792190.

### Butter(s) or Pasty Fatty Substances

For the purposes and within the meaning of this invention, the term “butter” (also alternatively termed “pasty fatty substances”) is used to refer to a lipophilic fatty compound with a reversible solid/liquid change of state and comprising a liquid fraction and a solid fraction at a temperature of 25° C. and at atmospheric pressure (760 mm Hg). In other words, the beginning of the melting point range of the pasty compound may be lower than 25° C. The liquid fraction of the pasty compound as measured at 25° C. may represent from 9% to 97% by weight of the compound. This liquid fraction at 25° C. represents preferably between 15% and 85%, still more preferably between 40% and 85% by weight. Preferably, the end of melting point range of the one or more butter(s) is lower than 60° C. Preferably, the one or more butter(s) has/have a hardness less than or equal to 6 MPa.

Preferably, the butters or pasty fatty substances have an anisotropic crystalline organisation in the solid state, which is visible by X-ray observations.

For the purposes and within the meaning of the invention, the melting point corresponds to the temperature of the most endothermic peak observed in thermal analysis (DSC) as described in the standard ISO 11357-3; 1999. The melting point of a paste or wax may be measured by means of a differential scanning calorimeter (DSC), for example the calorimeter sold under the trade name “DSC Q2000” by the company TA Instruments.

With regard to the measurement of the melting point range and the determination of the end of melting point range, the protocols for sample preparation and measurement are as described in the document WO2017046305.

The liquid fraction by weight of butter (or pasty fatty substances) at 25° C. is equal to the ratio of the enthalpy of fusion consumed at 25° C. to the enthalpy of fusion of the butter. The enthalpy of fusion of butter or pasty compound is the enthalpy consumed by the compound to pass from the solid state to the liquid state.

Butter is said to be in the solid state when the entirety of its mass is in crystalline solid form. Butter is said to be in the liquid state when the entirety of its mass is in liquid form. The enthalpy of fusion of butter is equal to the integral of the whole of the fusion curve obtained by using the requisite calorimeter, with a temperature rise of 5° C. or 10° C. per minute, in accordance with the standard ISO 11357-3:1999. The enthalpy of fusion of butter is the amount of energy necessary in order for the compound to change from a solid state to a liquid state. It is expressed in J/g.

The enthalpy of fusion consumed at 25° C. is the amount of energy absorbed by the sample in order for it to change from the solid state to the state that it exhibits at 25° C., constituted of a liquid fraction and a solid fraction. The liquid fraction of the butter as measured at 32° C. preferably represents from 30% to 100% by weight of the compound, preferably from 50% to 100%, even more preferably from 60% to 100% by weight of the compound. When the liquid fraction of the butter as measured at 32° C. is equal to 100%, the end of melting point range temperature of the pasty compound is less than or equal to 32° C. The liquid fraction of the butter as measured at 32° C. is equal to the ratio of the enthalpy of fusion consumed at 32° C. to the enthalpy of fusion of the butter. The enthalpy of fusion consumed at 32° C. is calculated in the same way as the enthalpy of fusion consumed at 23° C.

With regard to the measurement of hardness (firmness), the protocols for sample preparation and measurement are as described in in the patent document WO2017046305.

The pasty fatty substance or butter may be selected from among synthetic compounds and compounds of plant origin. A pasty fatty substance may be obtained by synthesis based on starting products of plant origin.

The pasty fatty substance is advantageously selected from among:
lanolin and its derivatives such as lanolin alcohol, oxyethylenated lanolins, acetylated lanolin, lanolin esters such as isopropyl lanolate, oxypropylenated lanolins;
vinyl polymers, in particular:
homopolymers of olefins;
copolymers of olefins;
hydrogenated homopolymers and copolymers of dienes;
homo- or copolymeric, linear or branched, oligomers of alkyl (meth)acrylates preferably having a C8-C30 alkyl group;
homo- and copolymeric oligomers of vinyl esters having C8-C30 alkyl groups;
homo- and copolymeric oligomers of vinyl ethers having C8-C30 alkyl groups;
fat- soluble polyethers resulting from the polyetherification between one or more C2-C100 diols, preferably C2-C50 diols;
esters and polyesters; and
mixtures thereof.

According to one preferred embodiment of the invention, the one or more particular butter(s) are of plant origin such as those described in Ullmann's Encyclopedia of Industrial Chemistry (“Fats and Fatty Oils”, A. Thomas, published 15 Jun. 2000, D01: 10.1002/14356007.a10_173, point 13.2.2.2. Shea Butter, Borneo Tallow, and Related Fats (Vegetable Butters)).

Particular mention should be made of C10-C18 triglycerides (INCI name: C10-18 Triglycerides) comprising a liquid fraction and a solid fraction at a temperature of and atmospheric pressure (760 mm Hg), shea butter, Nilotica shea butter (Butyrospermum parkii), Galam butter, (Butyrospermum parkii), Borneo butter or fat or tengkawang tallow (Shorea stenoptera), Shorea butter, Illipé butter, Madhuca or Bassia butter (Madhuca longifolia), Mowrah butter (Madhuca Latifolia), Katiau butter (Madhuca mottleyana), Phulwara butter (M. butyracea), mango butter (Mangifera indica), Murumuru butter (Astrocatyum murumuru), Kokum butter (Garcinia indica), Ucuuba butter (Virola sebifera), Tucuma butter, Painya butter (Kpangnan) (Pentadesma butyracea), coffee butter (Coffea arabica), apricot butter (Prunus Armeniaca), macadamia butter (Macadamia Temifolia), grape seed butter (Vitis vinifera), avocado butter (Persea gratissima), olive butter (Olea europaea), sweet almond butter (Prunus amygdalus dulcis), cocoa butter (Theobroma cacao) and sunflower butter, butter known by the INCI name Astrocaryum Murumuru Seed Butter, butter known by the INCI name Theobroma Grandiflorum Seed Butter, and butter known by the INCI name Irvingia Gabonensis Kernel Butter, jojoba esters (mixture of wax and hydrogenated jojoba oil) (INCI name: Jojoba esters), and shea butter ethyl esters (INCI name: Shea butter ethyl esters), and mixtures thereof.

According to one particularly preferred embodiment, the lipophilic gelling agent is selected from among Castor Oil/IPDI Copolymer (and) Caprylic/Capric Triglyceride, in particular marketed under the trade name Estogel M by the company Polymer Expert, Caprylic/Capric Triglyceride (and) Polyurethane-79, in particular marketed under the trade name OILKEMIA™ 5S polymer by the company Lubrizol, Trihydroxystearin, in particular marketed under the trade name THIXCIN® R by the company Elementis Specialties, and mixtures thereof, and more preferably Castor Oil/IPDI Copolymer (and) Caprylic/Capric Triglyceride.

According to one particular embodiment, a composition according to the invention, in particular the fatty phase, does not contain an elastomer gel comprising at least one dimethicone, in particular such as that marketed by the company NuSil Technology under the trade name CareSil™ CXG-1104 (INCI: Dimethicone (and) Dimethicone/Vinyl Dimethicone Crosspolymer).

Preferably, the viscosity of the fatty phase of a composition according to the invention is between 20 000 and 100 000 000 mPa·s, preferably between 50 000 and 1 000 000 mPa·s, and better still between 100 000 and 500 000 mPa·s, at 25° C.

Advantageously, a fatty phase of a composition according to the invention, in particular when it constitutes the dispersed phase, satisfies at least the following two physicochemical criteria of hardness and tackiness:
a hardness (x) of between 2 N and 14 N, preferably between 2.5 N and 12 N, in particular between 3 and 9 N, and better still between 4 and 6 N. The hardness (or firmness) corresponds to the maximum compression force as measured in Newtons divided by the surface area of the texturometer cylinder expressed in mm2 in contact with the sample being tested. In relation to a composition according to the invention, the hardness (x) is an indicator of the sensorial performance upon application to a keratinous material, in particular the skin. On the one hand, the hardness should not be too low in order to ensure that the droplets of dispersed fatty phase have sufficient mechanical resistance, in particular to shearing and/or to the mechanical stresses associated with, for example, the manufacture and packaging of the composition and/or the transportation thereof, and thus to ensure satisfactory kinetic stability, in particular in the presence of non-airless packaging. On the other hand, the hardness must not be excessively high so as not to impair the sensory properties, in particular the comfort and ease of application of the composition when applied to the skin. The greater the diameter of the droplets of dispersed fatty phase in a composition, the greater the extent to which the foregoing is exacerbated.
a tackiness (y) greater than or equal to −2 N, better still greater than or equal to −1 N, and in particular greater than or equal to −0.6 N. The tackiness (or adhesion (adhesiveness)) represents the work necessary in order to overcome the attractive forces between the surface of the product and the material with which it is in contact (for example, the total force necessary in order to separate the measuring tool from the sample). In relation to a composition according to the invention, the criterion of tackiness (y) is an indicator of the kinetic stability with regard to the phenomena of adhesion of the droplets to the wall of the packaging.

According to one embodiment, the fatty phase has a melting point of between 50° C. and 100° C., preferably between 60° C. and 90° C., and, at ambient temperature and atmospheric pressure, satisfies the following physicochemical criteria:
a hardness (x) of between 2 and 14 N, preferably between 2.5 and 12 N, better still between 3 and 9 N, and very particularly between 4 and 6 N; and
a tackiness (y) greater than or equal to −2 N, better still greater than or equal to −1 N, and in particular greater than or equal to −0.6 N; and/or
optionally, a cohesiveness (z) less than or equal to 40, preferably less than or equal to 35, and better still less than or equal to 30.

Preferably, a fatty phase of a composition according to the invention, particularly when it constitutes the dispersed phase, also has a cohesiveness (z) that is less than or equal to 40, preferably less than or equal to 35, and better still less than or equal to 30. Preferably, the dispersed fatty phase of a dispersion according to the invention advantageously has a cohesiveness (z) greater than or equal to 15, preferably greater than or equal to 20, and better still greater than or equal to 25. Advantageously, the dispersed fatty phase of a composition according to the invention has a cohesiveness (z) of between 15 and 40, preferably between 20 and 35, and better still between 20 and 30. The cohesiveness corresponds to the manner in which the product being tested resists the second deformation, relative to the manner in which it behaved during the first deformation. The cohesiveness corresponds to the area of the second curve (Area. 2) over the area of the first curve (Area 1) (i.e. Area. 2/Area. 1). In other words, the cohesiveness represents the forces within the sample being tested. Thus, strong bonds within the gel will provide for totally reversible deformation during the first compression, which will induce a force A2 that is identical to the force A1, and therefore 100% cohesiveness. Consequently, the stronger the cohesiveness, the more deformable the gel will be. The weaker the cohesiveness, the more brittle the gel will be (weak bonds, no resistance to stress). In relation to a composition according to the invention, the criterion of cohesiveness (z) is an indicator of kinetic stability with regard to the phenomena of the droplets of dispersed phase aggregating or even coalescing with one another. The cohesiveness corresponds to the property of the droplets whereby they adhere to each other. Thus, while a minimum level of cohesiveness is required so as to ensure the ‘gelled’ nature of the droplets, high cohesiveness is to be avoided in order to prevent the gelled droplets from adhering to each other.

The tools and methods for measuring hardness (firmness), adhesiveness and cohesiveness are described in the French patent application filed under the number FR2005408. Similarly as well, the experimental data relating to the criteria of hardness (x), tackiness (y) and cohesiveness (z) noted above are described in the application filed under the number FR2005408.

This combination of physicochemical criteria constitutes a non-obvious trade off characterising an anhydrous gel that is brittle but having very low tackiness and elasticity. As is clearly apparent from the examples provided in the application filed under the number FR2005408, this combination of physicochemical criteria makes it possible to access compositions in the form of macroscopic emulsions, in particular direct emulsions, that are endowed with particularly satisfactory, or indeed even improved performance levels, in terms of kinetic stability, and therefore of outcomes relating to visual and aesthetic appearance, sensory properties, and in particular comfort and ease of application on the skin, despite the lack of amodimethicone and therefore of membrane.

The selection of a lipophilic gelling agent that offers a natural index greater than 0.5, in particular greater than or equal to 0.75, better still greater than or equal to 0.85, and most particularly greater than or equal to 0.95 (according to the standard ISO 16128) falls within the scope of general knowledge of the person skilled in the art.

The person skilled in the art will strive to select the one or more lipophilic gelling agent(s) and/or the quantity thereof so as to satisfy the following: (i) a natural index greater than 0.5, preferably greater than 0.75, and better still greater than 0.95; and (ii) preferably in addition, the melting points and the physicochemical properties x and y, indeed even z, as mentioned above. In particular, the nature and/or the quantity of the one or more lipophilic gelling agent(s) must take into account the process (in particular of the “non-microfluidic” or “microfluidic” type) that is implemented for the manufacture of the dispersion according to the invention. These adjustments fall within the scope of competence and skill sets of the person skilled in the art with regard to the teaching in this description.

In particular, a composition according to the invention may comprise from 0.5% to 30%, preferably from 1% to 25%, in particular from 1.5% to 20%, better still from 2% to 15%, most particularly from 0.5% to 15%, and in particular from 5% to 12%, by weight of the one or more lipophilic gelling agent(s) relative to the total weight of the fatty phase that contains the latter.

Preferably, the content of the one or more lipophilic gelling agent(s) is greater than or equal to 2%, preferably greater than or equal to 5%, and better still greater than or equal to 8% by weight, relative to the total weight of the fatty phase that contains the latter. In a particularly advantageous manner, a composition according to the invention comprises the one or more lipophilic gelling agent(s) in content levels less than 20%, and preferably from 0.5% to 15%, by weight relative to the total weight of the fatty phase that contains the latter. Indeed, the Applicant has observed that using such content levels for the one or more lipophilic gelling agent(s) are advantageous in terms of sensory properties and comfort on application. This observation is notably illustrated through Example 4 of the international patent WO2021234135. The said Example 4 comprises 20% by weight of Rheopearl D2 relative to the total weight of the fatty phase and exhibits properties that are less satisfactory in terms of tackiness (or adhesiveness) and cohesiveness.

### Oil(s)

Advantageously, the fatty phase may comprise at least one oil, preferably one that offers a natural index greater than 0.5, and better still greater than or equal to 0.75, in particular greater than or equal to 0.85, and most particularly greater than or equal to 0.95 (according to the standard ISO 16128).

The term “oil” is understood to refer to a fatty substance which is liquid at ambient temperature and atmospheric pressure.

By way of oils according to the invention, the following may be mentioned as examples:
hydrocarbon oils of plant origin, as described here below;
hydrocarbon oils of animal origin, such as perhydrosqualene and squalane;
synthetic esters and ethers, in particular of fatty acids, such as oils having the formulas R₁COOR₂ and R1 OR₂, in which R₁ represents the residue of a C₈ to C₂₉ fatty acid, and R₂ represents a C₃ to C₃₀ hydrocarbon chain, either branched or unbranched, such as for example Purcellin oil, isononyl isononanoate, isodecyl neopentanoate, isostearyl neopentanoate, isopropyl myristate, octyldodecyl myristate, 2-ethyl- hexyl palmitate, 2-octyl- dodecyl stearate, 2-octyl- dodecyl erucate, and isostearyl isostearate; hydroxy esters such as isostearyl lactate, octylhydroxystearate, octyldodecyl hydroxystearate, diisostearyl malate, triisocetyl citrate, heptanoates, octanoates, decanoates of fatty alcohols; polyol esters such as propylene glycol dioctanoate, neopentyl glycol diheptanoate, and diethylene glycol diisononanoate; and pentaerythritol esters such as pentaerythrityl tetrabenate (DUB PTB) or pentaerythrityl tetraisostearate (Prisorine 3631);
linear or branched hydrocarbons of inorganic/mineral or synthetic origin, such as volatile or non-volatile paraffin oils, and the derivatives thereof, petroleum jelly, polydecenes, hydrogenated polyisobutene such as Parléam oil;
fatty alcohols having from 8 to 26 carbon atoms, such as cetyl alcohol, stearyl alcohol and the mixture thereof (cetyl stearyl alcohol), or octyldodecanol;
and mixtures thereof.

Preferably, the fatty phase of a dispersion according to the invention comprises at least one plant oil.

By way of hydrocarbon oil(s) of plant origin, mention may be made of triglycerides of caprylic and capric acids, triglycerides of caprylic, capric (also known by the term “MCT oil”), myristic and stearic acids (INCI name: Caprylic/capric/myristic/stearic Triglyceride), triethylhexanoin, oil of limnanthes seeds Limnanthes Alba (INCI name: Limnanthes Alba (Meadowfoam) Seed Oil), macadamia nut oil (INCI name: Macadamia Ternifolia Seed Oil), Rosa Canina rosehip oil (INCI name: Rosa Canina Fruit Oil), soya oil (INCI name: Glycine Soja (Soybean) Oil), sunflower seed oil (INCI name: Helianthus Annuus (Sunflower) Seed Oil), tribehenin (INCI name: tribehenin), triisostearin (INCI name: triisostearin), apricot kernel oil (INCI name: Prunus Armeniaca (Apricot) Kernel Oil), rice bran oil (INCI name: Oryza Sativa (Rice) Bran Oil), argan oil (INCI name: Argania Spinosa Kernel Oil), avocado oil (INCI name: Persea Gratissima Oil), evening primrose oil (INCI name: Oenothera Biennis Oil), rice germ oil (INCI name: Oryza Sativa Germ Oil), Hydrogenated Coconut Oil (INCI name: Hydrogenated Coconut Oil), sweet almond oil (INCI name: Prunus Amygdalus Dulcis Oil), sesame seed oil (INCI name: Sesamum Indicum Seed Oil), hydrogenated rapeseed oil (INCI name: Hydrogenated Rapeseed Oil), safflower seed oil (INCI name: Carthamus Tinctorius Seed Oil),

Queensland Macadamia integrifolia nut oil (INCI name: Macadamia Integrifolia Seed Oil), tricaprylin (or triacylglycerol), wheat germ oil (INCI name: Triticum Vulgare Germ Oil), borage seed oil (INCI name: Borago Officinalis Seed Oil), Shea Butter Oil (INCI name: Butyrospermum Parkii Oil), hydrogenated Castor Oil (INCI name: Hydrogenated Castor Oil), Chinese cabbage seed oil (INCI name: Brassica Campestris Seed Oil), Camellia oil, and in particular Japanese Camellia seed oil (INCI name: Camellia Japonica Seed Oil), green tea seed oil (INCI name: Camellia Sinensis Seed Oil), sea buckthorn oil (INCI name: Hippophae Rhamnoides Oil), Camellia Kissi seed oil (INCI name: Camellia Kissi Seed Oil), Moringa seed oil (INCI name: Moringa Pterygosperma Seed Oil), canola oil (INCI name: Canola Oil), tea seed oil (INCI name: Camellia Oleifera Seed Oil), carrot seed oil (INCI name: Daucus Carota Sativa Seed Oil), triheptanoin (INCI name: Triheptanoin), vanilla oil (INCI name: Vanilla Planifolia Fruit Oil), glycerides of canola oil and phytosterols (INCI name: Phytosteryl Canola Glycerides), blackcurrant seed oil (INCI name: Ribes Nigrum (Black Currant) Seed Oil), karanja seed oil (INCI name: Pongamia Glabra Seed Oil), roucou or annatto oil (INCI name: Roucou (Bixa orellana) Oil), and mixtures thereof.

Advantageously, the fatty phase comprises at least one oil having a refractive index close to that of the aqueous continuous phase, that is to say an oil having a refractive index, at ambient temperature and atmospheric pressure, which is preferably between 1.2 and 1.6, preferably between 1.25 and 1.5, and in particular between 1.3 and 1.4. This embodiment is advantageous in that it provides the means to enhance the transparency of the fatty phase, and therefore the transparency of the dispersion according to the invention. The transparency may be qualified according to the method described in the document WO2018/167309.

Advantageously, the fatty phase of a composition according to the invention comprises at least one, or even at least two, oil(s), preferably selected from among hydrocarbon oil(s) of plant origin, and preferably selected from among Limnanthes Alba limnanthes seed oil (INCI name: Limnanthes Alba (Meadowfoam) Seed Oil, triglycerides of caprylic, capric acids, and mixtures thereof.

Preferably, a composition according to the invention comprises a content level of less than 1%, and better still less than 0.5%, or indeed comprises no amount, of oil(s) having a natural index according to the standard ISO 16128 that is less than 0.5, preferably less than 0.75, and better still less than 0.95.

Preferably, a composition according to the invention comprises a content level of less than 1%, and better still less than 0.5%, or indeed comprises no amount, of silicone oil or fluorinated oil.

The selection of oils having a natural index greater than 0.5, in particular greater than or equal to 0.75, better still greater than or equal to 0.85, and most particularly greater than or equal to 0.95 (according to the standard ISO 16128) falls within the scope of general knowledge of the person skilled in the art.

Advantageously, the person skilled in the art will strive to select the one or more oil(s) and/or the quantity thereof in a manner so as to satisfy the criteria of melting points, and physicochemical properties x and y, indeed even z, as mentioned above, of the fatty phase. These adjustments fall within the scope of competence and skill sets of the person skilled in the art with regard to the teaching in this description.

A composition according to the invention may comprise between 10% and 99.5%, preferably between 20% and 90%, better still between 30% and 85%, and in particular between 50% and 80%, by weight of the one or more oil(s) relative to the total weight of the fatty phase that contains the latter.

A composition according to the invention may comprise from 1% to 50%, preferably from 5% to 40%, and better still from 10% to 25%, by weight of the one or more oil(s) relative to the total weight of the said composition.

Contrary to all expectations, the inventors have observed that a composition according to the invention remains satisfactory in terms of kinetic stability even where high percentages of dispersed phase are present, in particular when the composition is a direct emulsion. Thus, a composition according to the invention may advantageously comprise from 1% to 60%, in particular from 5% to 50%, preferably from 10% to 40%, and better still from 15% to 30%, by weight of dispersed phase, preferably of dispersed fatty phase or dispersed aqueous phase, relative to the total weight of the composition.

### Additional Compound(s)

A composition according to the invention, in particular the aqueous phase and/or the fatty phase, may furthermore also comprise at least one additional compound other than the lipophilic gelling agent and the oils mentioned above.

Preferably, the one or more additional compound(s) is/are selected from the additional compounds that offer a natural index greater than or equal to 0.5, in particular greater than or equal to 0.75, and better still greater than or equal to 0.95 (according to the standard ISO 16128). Effecting this selection falls within the scope of general knowledge of the person skilled in the art

By way of an additional compound, a composition according to the invention, in particular the aqueous phase and/or the fatty phase, may also comprise powders; fillers; flakes; colouring agents, in particular selected from among water-soluble or water-insoluble, fat-soluble or fat-insoluble, organic or inorganic colouring agents, optical effect materials, liquid crystals, and mixtures thereof; particulate agents that are insoluble in the fatty phase; preservatives; humectants; fragrancing agents, in particular as defined in the document WO2019002308; stabilisers; chelating agents; emollients; modifying agents chosen from gelling/texture agents, viscosity agents other than the base and lipophilic gelling agents mentioned above, pH, osmotic strength and/or refractive index modifiers, etc, or any customary cosmetic additive; and mixtures thereof.

For the purposes and within the meaning of the invention, the term “filler” is used to refer to colourless or white solid particles of any shape, which are present in an insoluble form and dispersed in the medium of the composition. They are inorganic or organic in nature and serve the purpose of providing body or rigidity and/or softness and uniformity to the deposit, particularly in the context of make-up, and enhanced stability with regard to exudation and the properties of non-migration after application, and/or mattness, and/or coverage.

For the purposes and within the meaning of the invention, the term “particulate agents insoluble in the fatty phase” is used to refer to the group constituted of pigments, ceramics, polymers, in particular acrylic polymers, and mixtures thereof.

By way of additional compound, a composition according to the invention, in particular the aqueous phase and/or the fatty phase, may also comprise at least one biological/cosmetic active ingredient, in particular selected from among moisturising agents, healing agents, depigmenting agents, UV filters, desquamating agents, antioxidising agents, active ingredients that stimulate the synthesis of dermal and/or epidermal macromolecules, dermodecontracting agents, antiperspirant agents, soothing agents, and/or anti-ageing agents, and mixtures thereof. Such active ingredients are described in particular in the French patent FR1558849.

### Hydrophilic Gelling Agent(s)

Advantageously, the aqueous phase may also comprise at least one hydrophilic gelling agent, that is to say, one that is soluble or dispersible in water. In the context of the present invention, the term “hydrophilic gelling agent” may be used interchangeably with the term “hydrophilic texture agent”. The hydrophilic gelling agents serve to enable modulating of the fluidity of the dispersion, and therefore of the sensorial and/or galenic properties which it is desired to obtain, and/or contribute to further improving the kinetic stability of the dispersion.

By way of hydrophilic gelling agents, mention may be made of:
natural gelling agents, in particular selected from algae extracts, plant exudates, seed extracts, microorganism exudates, such as alkasealan marketed by the company Hakuto (INCI: Alcaligenes Polysaccharides), and other natural agents, in particular hyaluronic acid;
semi-synthetic gelling agents, in particular selected from among cellulose derivatives and modified starches;
synthetic gelling agents, in particular selected from among homopolymers of (meth)acrylic acid or one of the esters thereof, copolymers of (meth)acrylic acid or one of the esters thereof, copolymers of AMPS (2-acrylamido -2- methylpropanesulphonic acid), associative polymers;
other gelling agents, in particular selected from clays, glycerine; and
mixtures thereof.

For the purposes and within the meaning of the present invention, the term “associative polymer” is used to refer to any amphiphilic polymer containing in its structure at least one fatty chain and at least one hydrophilic portion; the associative polymers that are in accordance with the present invention may be anionic, cationic, nonionic or amphoteric; they include, in particular, those described in the French patent FR2999921. Preferably, they are the amphiphilic and anionic associative polymers and the amphiphilic and non-ionic associative polymers as described herein below.

These hydrophilic gelling agents are described in greater detail in the French patent FR3041251.

According to one embodiment, a composition according to the invention comprises from 0.0001% to 20%, preferably from 0.001% to 15%, in particular from 0.01% to 10%, and better still from 0.1% to 5%, by weight of one or more hydrophilic gelling agent(s) relative to the total weight of the aqueous phase that contains the latter. According to one embodiment, the composition according to the invention comprises from 0.0001% to 30%, preferably from 0.001% to 20%, in particular from 0.01% to 10%, and better still from 0.1% to 5%, by weight of one or more hydrophilic gelling agent(s) relative to the total weight of the aqueous phase that contains the latter.

According to one embodiment, the composition according to the invention comprises from 0.0001% to 20%, preferably from 0.001% to 15%, and preferentially from 0.01% to 10%, by weight of the one or more additional compound(s) relative to the total weight of the said composition.

A composition according to the invention may comprise oils, gelling agents, and/or one or more additional compound(s) that have a natural index of zero (according to the standard ISO 16128), with the proviso that the percentage of ingredients of natural origin in the final composition remains greater than or equal to 95% according to the standard ISO 16128.

By way of illustration, the gelling agents that have a natural index of zero (according to the standard ISO 16128) may be selected from among:
elastomeric organopolysiloxanes that are partially or totally crosslinked, and having a three-dimensional structure;
polyacrylates, for example resulting from the polymerisation of one or more: C10-C30 alkyl acrylate(s), preferably C14-C24 alkyl acrylate(s), and even more preferably C18-C22 alkyl acrylate(s);
silicone polyamides of the polyorganosiloxane type such as those described in the patents U.S. Pat. Nos. 5,874,069, 5,919,441, 6,051,216 and 5,981,680;
silicone waxes, which can advantageously be substituted polysiloxanes, preferably with a low melting point;
hydrocarbon waxes modified with silicone or fluoro groups, for example: siliconyl candelilla, siliconyl beeswax, and Koster Keunen Fluorobeeswax;
fluorinated waxes;
polymeric or non-polymeric silicone compounds such as high molecular weight polydimethylsiloxanes, polydimethylsiloxanes with side chains of alkyl or alkoxy type having from 8 to 24 carbon atoms, in particular stearyl dimethicones;
polymeric or non-polymeric fluorinated compounds; and
mixtures thereof.

By way of illustration, oils having a natural index of zero (according to the standard ISO 16128) may be selected from among:
silicone oils, such as, for example, volatile or non-volatile polymethylsiloxanes (PDMS) having a linear or cyclic silicone chain, which are liquid or pasty at ambient temperature, in particular cyclopolydimethylsiloxanes (cyclomethicones) such as cyclohexasiloxane and cyclopentasiloxane; polydimethylsiloxanes (or dimethicones) containing alkyl, alkoxy or phenyl groups, either pendant or at the end of the silicone chain, these groups having from 2 to 24 carbon atoms; phenylated silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
fluorinated oils that are partially hydrocarbon-based and/or silicone-based, such as those described in the document JP-A-2-295912; and
mixtures thereof.

The person skilled in the art will know how to adjust the content of compounds having a natural index of zero (according to the standard ISO 16128) in a manner such that the percentage of ingredients of natural origin in the final composition remains greater than or equal to 95% according to the standard ISO 16128.

In particular, the nature and/or the quantity of the one or more additional compound(s) depend(s) on the aqueous or fatty nature of the phase under consideration, of the composition according to the invention, and/or must take into account the process (in particular of the “non-microfluidic” or “microfluidic” type) that is implemented for the manufacture of the composition according to the invention. Making these choices and adjustments falls within the scope of competence and skill sets of the person skilled in the art.

Advantageously, the person skilled in the art will strive to select the one or more possible additional compound(s) and/or the quantity thereof in a manner such that the advantageous properties of the dispersion according to the invention, in particular its kinetic stability and preferably, with respect to the dispersed fatty phase, its melting point and its physicochemical properties x and y, or even z, mentioned above, are not or are substantially not altered by the envisaged addition.

### Method of Preparation

A composition according to the invention may be prepared by means of various processes.

For example, a composition according to the invention offers the advantage that it can be prepared according to a simple “non-microfluidic” process, that is to say by simple emulsification, in particular by using an agitation device such as a Rayneri-type agitator or a paddle agitator.

As in a conventional emulsion, an aqueous solution and a fatty solution are prepared separately.

For example, in the case of a direct emulsion, it is the addition under agitation of the fatty phase to the aqueous phase that creates the said direct emulsion. The viscosity of the aqueous phase can be controlled, in particular, by making adjustments to the quantity of hydrophilic gelling agent and/or the pH of the solution. As a general rule, the pH of the aqueous phase is lower than 4.5, which may involve the addition of a third viscosity-increasing solution, in particular a soda solution (or base, or (BF)) at a later stage in order to achieve a pH of between 5.5 and 6.5.

The viscosity of the phases, in particular that of the continuous phase, and the shear force applied to the mixture are the two main parameters that influence the size and monodispersity of the droplets in the emulsion.

The person skilled in the art will be able to adjust the parameters of the non-microfluidic method in order to obtain a composition according to the invention, and in particular to satisfy the desired diameter related criterion for the droplets of the dispersed phase.

A composition according to the invention may also be prepared according to a microfluidic method, in particular as described in the international patent applications WO2012/120043, WO2015055748, or WO2019/145424. According to this embodiment, the one or more microfluidic nozzle(s) used in implementation may have a configuration based on the T-shaped geometry, co-flow (or co-currents), or flow-focusing geometries.

According to this embodiment, the droplets obtained by this microfluidic method advantageously exhibit a uniform size distribution.

Preferably, the compositions of the invention are constituted of a population of monodisperse droplets, in particular such that they have an average diameter D̅ ranging from 100 μm to 3,000 μm, in particular from 500 μm to 3,000 μm, and a coefficient of variation Cv that is less than 10%, preferably less than or equal to 5%, or indeed even less than or equal to 3%.

In the context of this description, the term “monodisperse droplets” is used to refer to the fact that the population of droplets of the composition according to the invention has a uniform size distribution. The monodisperse droplets exhibit good monodispersity. Conversely, droplets exhibiting poor monodispersity are referred to as “polydisperse”.

According to one embodiment, the average diameter D̅ of the droplets is measured, for example, by analysing a photograph of a batch consisting of N droplets, using a software application for image (Image J) processing. Typically, based on this method, the diameter is measured in pixels and then reported in μm, as a function of the size of the container containing the droplets of the dispersion.

Preferably, the value of N is chosen so as to be greater than or equal to 30, advantageously greater than or equal to 100, in a manner such that this analysis reflects in a statistically significant manner the distribution of diameters of the droplets of the said emulsion.

The diameter Di of each droplet is measured, and then the average diameter D̅ is obtained by calculating the arithmetic mean of these values:

$$\overline{D} = \frac{1}{N} {\sum }_{i = 1}^{N} {D}_{i}$$

Based on these values Di, it is also possible to obtain the standard deviation σ of the diameters of the droplets of the dispersion:

$$σ = \sqrt{\frac{{\sum }_{i = 1}^{N} {( {D}_{i} - \overline{D} )}^{2}}{N}}$$

The standard deviation σ of a dispersion reflects the distribution of the diameters Di of the droplets of the dispersion around the average diameter D̅.

By knowing the average diameter D̅ and the standard deviation σ of a dispersion, it can be determined that 95.4% of the droplet population are to be found in the interval of diameters [D̅−2σ; D̅+2σ] and that 68.2% of the population are to be found in the interval [D̅−σ; D̅+σ].

In order to characterise the monodispersity of the dispersion according to this embodiment of the invention, the coefficient of variation may be calculated as follows:

$${C}_{ν} = \frac{σ}{\overline{D}}$$

This parameter reflects the distribution of the diameters of the droplets as a function of the average diameter D̅ of the latter.

The coefficient of variation Cv of the diameters of the droplets according to this embodiment of the invention is less than 10%, preferably less than or equal to 5%, or indeed even less than or equal to 3%.

Alternatively, the monodispersity may be effectively demonstrated by placing a dispersion sample in a flask having a constant circular cross-section. Gentle agitation by rotating a quarter turn for half a second about the axis of symmetry passing through the flask, followed by a rest of half a second is carried out, before repeating the operation in the opposite direction, to be performed four times in succession.

The droplets of the dispersed phase are organised in a crystalline form when they are monodispersed. As a result, they appear to be packed according to a pattern that repeats itself in three dimensions. It is thus possible to observe a regular packing, which indicates good monodispersity, and an irregular packing, which reflects the polydispersity of the composition.

In order to obtain monodisperse droplets, it is also possible to use in implementation the microfluidic technique (Utada et al. MRS Bulletin 32, 702-708 (2007); Cramer et al. Chem. Eng. Sci. 59, 15, 3045-3058 (2004)), and more particularly microfluidic devices of the co-flow type (the fluids flow in the same direction) or flow-focusing type (the fluids flow in different directions, and typically in opposite directions).

The method for preparing a composition according to the invention includes at least the following steps:
a) providing at least one oily fluid FH in liquid form and at least one aqueous fluid FA in liquid form, these being immiscible with each other at ambient temperature and atmospheric pressure;
b) injecting the aqueous fluid FA into the oily fluid FH under agitation, or vice versa, in order to obtain an emulsion comprising droplets of dispersed phase, constituted of the oily fluid FH or the aqueous fluid FA, in a continuous phase constituted of the other of the aqueous fluid FA or the oily fluid FH;
c) optionally, cooling the composition obtained in the step (b) to ambient temperature or to a temperature lower than ambient temperature, and
d) recovering the composition in emulsion form.

Preferably, the method for preparing a composition according to the invention is based on a microfluidic method and may then include at least the following steps:
a) providing at least one oily fluid FH in liquid form and at least one aqueous fluid FA in liquid form, which are immiscible with each other at ambient temperature and atmospheric pressure;
b) injecting the aqueous fluid FA and the oily fluid FH into a microfluidic device that is capable of forming droplets of dispersed phase, constituted of the oily fluid FH or the aqueous fluid FA, in which the continuous phase, constituted of the other of the aqueous fluid FA or the oily fluid FH, circulates continuously;
c) optionally, cooling the composition obtained in the step (b) to ambient temperature or to a temperature lower than ambient temperature; and
d) continuously recovering the composition in emulsion form.

According to one embodiment, the oily fluid FH is initially prepared by mixing a fatty phase comprising at least one lipophilic gelling agent and optionally at least one oil and further, on an optional basis, at least one additional compound as mentioned above.

According to one embodiment, the aqueous fluid FA is initially prepared by mixing an aqueous phase with, on an optional basis, at least one base, at least one additional compound, preservatives, and/or other water-soluble products such as glycerine, and most particularly at least one hydrophilic gelling agent.

According to a first embodiment, the aqueous fluid FA is intended to constitute the continuous phase and the oily fluid FH is intended to constitute the dispersed phase.

According to one other particular embodiment, the aqueous fluid FA is intended to constitute the dispersed phase and the oily fluid FH is intended to constitute the continuous phase.

When the aqueous fluid FA and/or the oily fluid FH comprises at least one gelling agent, the steps (a) and (b) are advantageously carried out at a temperature higher than or equal to the melting point of the one or more gelling agent(s) used, and preferably at a temperature higher than or equal to the highest melting point of the gelling agents used in implementation. In other words, the steps (a) and (b) are carried out with an oily fluid FH in a form that is capable of emulsifying with the aqueous fluid FA and therefore capable of ensuring the formation of droplets, and therefore with an aqueous fluid FA and an oily fluid FH in liquid form.

The presence, in the dispersed phase, or indeed even in the continuous phase, of one or more gelling agent(s) may necessitate adjustments to the composition preparation method for preparing a composition according to the invention. In particular, the method for preparing such a composition according to the invention includes a step of heating (between 50° C. and 150° C., in particular between 60° C. and 90° C.) at least the fatty phase prior to mixing/bringing into contact the said fatty phase with the aqueous phase (or vice versa) and, where appropriate, the maintaining of this heating (i) during the agitation in the case of a “non-microfluidic” process; or (ii) at the level of the microfluidic device in the case of a “microfluidic” process, until such time as the desired composition is obtained.

Preferably, in a method according to the invention, the step (a) includes:
a1) optionally, the heating of an oily fluid FH, to a temperature ranging from 50° C. to 150° C., preferably from 60° C. to 120° C., and better still from 70° C. to 100° C.; and/or
a2) optionally, the heating of an aqueous fluid FA, to a temperature ranging from 50° C. to 150° C., preferably from 60° C. to 120° C., and better still from 70° C. to 100° C.

Preferably, in a method according to the invention, the step (b) is carried out at a temperature ranging from 50° C. to 150° C., preferably from 60° C. to 120° C., and better still from 70° C. to 100° C.

Advantageously, a method according to the invention includes the aforementioned step (c) of cooling. This step (c) advantageously makes it possible to accelerate the cooling kinetics of the composition formed, and thus prevent the risks of coalescence and fragmentation of the droplets post-formation (between 10 and 30° C.).

In particular, the step (c) consists of a cooling step for cooling the composition which is carried out at a temperature that is lower than the melting point of the one or more gelling agent(s) used in implementation, and preferably at a temperature that is lower than the lowest among the melting points of the gelling agent(s) used in implementation. In particular, the step (c) consists of a cooling step carried out at a temperature that is lower than ambient temperature. In particular, the step (c) is carried out at a temperature ranging from 0° C. to 25° C., preferably from 5° C. to 20° C., and better still from 10° C. to 15° C. This step (c) may be based on the passing of the composition through a multitubular exchanger, preferably mounted in the immediate vicinity of the one or more outlet(s) of the microfluidic device.

Advantageously, the oily fluid FI, in particular when intended to constitute the dispersed fatty phase of a composition according to the invention, comprises at least one lipophilic gelling agent and optionally at least one oil and has a melting point of between 50° C. and 100° C., preferably between 60° C. and 90° C., and, at ambient temperature and atmospheric pressure, satisfies the following physicochemical criteria:
a hardness (x) of between 2 and 14 N, preferably between 2.5 and 12 N, better still between 3 and 9 N, and most particularly between 4 and 6 N; and
a tackiness (y) greater than or equal to −2 N, better still greater than or equal to −1 N, and in particular greater than or equal to −0.6 N;
the oily fluid FH moreover also being free of amodimethicone and, optionally, also comprising at least one additional compound as mentioned above; and
the aqueous fluid FA comprises at least water and, optionally, at least one additional compound as mentioned above, and preferably at least one hydrophilic gelling agent.

In the case of a “non-microfluidic” process as mentioned above, the step (b) is constituted by an agitation process in which the heating may be maintained during this agitation which serves the purpose of obtaining the desired composition.

In the case of a “microfluidic” process as mentioned above, the microfluidic device as such may be adapted so as to be maintained at a temperature ranging from to 150° C., preferably from 80° C. to 90° C.

In the case of a “microfluidic” process, the step (b) of formation of droplets may include the formation of droplets of a first fluid from the oily fluid FH or the aqueous fluid FA at the outlet of a first conduit opening into the other fluid from the oily fluid FH or the aqueous fluid FA. Preferably, the fluid dedicated to forming the continuous phase is circulated through a second conduit, the outlet of the first conduit opening into the second conduit, advantageously coaxially with the local axis of the second conduit.

According to one embodiment in which the aqueous fluid FA is intended to constitute the continuous phase, the manufacturing method for manufacturing a dispersion according to the invention may further include, between the steps (b) and (d), or even between the steps (b) and (c) when present, a step (e) of injecting a viscosity-increasing solution as previously defined above, for example as described in the document WO2015/055748. This viscosity-increasing solution is typically injected into the aqueous fluid FE after formation of the composition according to the invention, and therefore after formation of the droplets. According to one embodiment, the viscosity-increasing solution comprises at least one base, in particular an alkali hydroxide, such as sodium hydroxide.

The present invention also relates to a composition that is able to be obtained by a method such as those described here above.

### Uses

In a preferred manner, a composition according to the invention can be used directly, on conclusion of the abovementioned preparation processes, by way of a composition, in particular a cosmetic composition. The dispersion according to the invention, when prepared by means of a microfluidic method as described here above, can also be used as a composition, in particular a cosmetic composition, after separation of the droplets and redispersion thereof in an appropriate second phase.

The invention also relates to the use of a composition according to the invention for the preparation of a composition, in particular a cosmetic, pharmaceutical, nutritional, or agri-food composition.

Preferably, the present invention relates to a cosmetic composition, in particular a care and/or make-up composition, for a keratinous material, in particular for the skin and/or the hair, and more particularly for the skin. When the composition is in the form of a direct emulsion, the aqueous continuous phase may also comprise at least one physiologically acceptable medium.

The physiologically acceptable medium is generally appropriate to the nature of the support over which the composition is to be applied, as well as to the finished appearance of the composition in which it is to be packaged.

In the context of the invention, and unless otherwise stated, the term “physiologically acceptable medium” is used to refer to a medium that is appropriate for cosmetic applications, and in particular appropriate for the application of a composition of the invention to a keratinous material, in particular the skin and/or the hair, and more particularly the skin. According to one embodiment, the physiologically acceptable medium is represented directly by the aqueous continuous phase as described here above.

The cosmetic compositions of the invention may be, for example, a cream, a lotion, a serum, and a gel for the skin (hands, face, feet, etc.), a foundation (liquid, paste), a preparation for baths and showers (salts, foams, oils, gels, etc.), a hair care product (hair dyes and bleaches), a cleansing product (lotions, powders, shampoos), a hair care product (lotions, creams, oils), a hair styling product (lotions, lacquers, glosses), a shaving product (soaps, foams, lotions, etc.), a product intended to be applied to the lips, a suncare product, a sunless tanning product, a product for whitening the skin, an anti-wrinkle product. In particular, the cosmetic compositions of the invention may be an anti-ageing serum, a rejuvenating serum, a moisturising serum or a perfumed water.

Thus, in view of the foregoing, a composition according to the invention is oral or topical, preferably topical, and better still topical on a keratinous material, in particular the skin, and better still the skin of the face.

The present invention also relates to a non-therapeutic process for the cosmetic treatment of a keratinous material, in particular the skin and/or the hair, and more particularly the skin, which includes a step of applying to the said keratinous material at least one aforementioned cosmetic composition.

The present invention also relates to the use of a composition according to the invention, in order to enhance the surface appearance of the skin, in particular in order to moisturise the skin and/or reduce fine lines and wrinkles.

Throughout the description, the expression “comprising a” is to be understood as being synonymous with the expression “comprising at least one”, unless otherwise specified. The expressions “between . . . and . . . ”, “from . . . to . . . ”, and “ ranging from . . . to . . . ” are to be understood as being inclusive, unless the contrary is specified. The quantities of the ingredients appearing in the examples are expressed as a percentage by weight relative to the total weight of the composition, unless otherwise specified.

The examples following here below illustrate the present invention without in any way limiting the scope thereof.

### EXAMPLES

### Example 1: Calculation of a Percentage of Ingredients of Natural Origin According to the Standard ISO 16128

The purpose of example 1 is to shed light on the calculation method for calculating the percentage of ingredients of natural origin according to the standard ISO 16128 based on the theoretical formula described in table 1 here below.

In order to calculate the percentage of ingredients of natural origin, simply multiply the percentage of the ingredient in the formula with its natural index (a value generally provided by the manufacturer or supplier of the ingredient). For example, for a shower gel with argan oil described in Table 1 above, the percentage of ingredients of natural origin is 95.5%. In this case, it would be possible to indicate “NATURAL product” on the packaging.

### Example 2: Topical Macroscopic Direct Emulsions for Skin Care

Two compositions in the form of a macroscopic direct emulsion are prepared by using a microfluidic method as described in the patent document WO2015055748, that is to say a comparative composition 2A corresponding to Example 2A of the patent application WO2017046305 and a composition 2B according to the invention. The formulas for the continuous aqueous phases and the dispersed fatty phases respective to these two compositions 2A and 2B are described in Tables 2A and 2B here below.

TABLE 2A
Comparative Composition 2A according to Example 2 of the
Document WO2017046305:
COMPARITIVE FINAL COMPOSITION 2A

| | | | | | |
|---|---|---|---|---|---|
| | | % m | | | |
| | | in the | Final | Natural | % |
| Trade name | INCI name | phase | % m | index* | Natural* |

Aqueous phase (OF)

| | | | | | |
|---|---|---|---|---|---|
| Reverse Osmosis Water | Aqua | 75.147 | 69.06 | 1 | 69.06 |
| Glycerine codex | Glycerine | 16.152 | 14.844 | 1 | 14.844 |
| Zemea propanediol | Propanediol | 5.384 | 4.948 | 1 | 4.948 |
| Microcare PTG | Pentylenglycol | 2.146 | 1.972 | 0 | 0 |
| Microcare PE | Phenoxyethanol | 0.858 | 0.789 | 0 | 0 |
| Carbomer Tego | Carbomer | 0.233 | 0.214 | 0 | 0 |
| 340FD | | | | | |
| EDETA | Disodium EDTA | 0.037 | 0.034 | 0 | 0 |
| Sodium hydroxyde | Sodium hydroxyde | 0.043 | 0.039 | 0 | 0 |
| pellets PRS codex | | | | | |
| Total | | 100 | 91.900 | — | — |

Fatty Phase (IF)

| | | | | | |
|---|---|---|---|---|---|
| DUB ININ | Isononyl | 89.49 | 7.24819 | 0 | 0 |
| | isononanoate | | | | |
| Rheopearl KL2 | Dextrin palmitate | 10 | 0.810 | 1 | 0.81 |
| KF 8004 | Amodiméthicone | 0.5 | 0.041 | 0 | 0 |
| Phat Blue DC 6204 | CI 61565. | 0.01 | 0.00081 | 0 | 0 |
| | CI 60725 | | | | |
| Total | | 100 | 8.100 | — | 89.662 |

*according to the standard ISO 16128.

TABLE 2B
Composition 2B according to the Invention:
FINAL COMPOSITION 2B

| | | | | | |
|---|---|---|---|---|---|
| | | % w | | | |
| | | in the | Final | Natural | % |
| Trade name | INCI name | phase | % w | index* | Natural* |

Aqueous phase (OF)

| | | | | | |
|---|---|---|---|---|---|
| Eau osmosée | Aqua | 75.147 | 69.06 | 1 | 69.06 |
| [Osmosis water] | | | | | |
| Glycerine codex | Glycerine | 16.152 | 14.844 | 1 | 14.84 |
| Zemea | Propanediol | 5.384 | 4.948 | 1 | 4.95 |
| propanediol | | | | | |
| PENTIOL | PENTYLENE | 2.146 | 1.972 | 1 | 1.97 |
| GREEN+/A- | GLYCOL. AQUA | | | | |
| LEEN 5 | | | | | |
| PURECARE | CHLORPHENESIN | 0.858 | 0.789 | 0 | 0 |
| CHP XO | | | | | |
| Carbomer | Carbomer | 0.233 | 0.214 | 0 | 0 |
| Tego 340FD | | | | | |
| EDETA | Disodium EDTA | 0.037 | 0.034 | 0 | 0 |
| Sodium | Sodium hydroxyde | 0.043 | 0.039 | 0 | 0 |
| hydroxyde | | | | | |
| pellets PRS | | | | | |
| codex | | | | | |
| Total | | 100 | 91.900 | — | — |

Fatty Phase (IF)

| | | | | | |
|---|---|---|---|---|---|
| Meadowfoam | Limnanthes | 22.61 | 1.83 | 1 | 1.83 |
| Oil | alba seed oil | | | | |
| CETIOL C5 C | Coco- | 62.38 | 5.05 | 1 | 5.05 |
| | Caprylate/Caprate | | | | |
| Rheopearl | Dextrin palmitate | 15 | 1.215 | 1 | 1.215 |
| KL2 | | | | | |
| Phat Blue DC | CI 61565. | 0.01 | 0.00081 | 0 | 0 |
| 6204 | CI 60725 | | | | |
| Total | | 100 | 8.100 | — | 98.915 |

*according to the standard ISO 16128.

The composition 2B differs essentially from the comparative composition 2A in terms of (i) the absence of amodimethicone, (ii) the presence of oily solvents, which are incompatible as such in the composition 2A in view of their inability to solubilise amodimethicone in a satisfactory manner, and of humectants, all of which have high natural indexes, and (iii) an increase in the lipophilic gelling agent content.

Protocol for OF and IF preparation: the preparation protocols for preparing OF and IF fall within the scope of general knowledge of the person skilled in the art. The solutions obtained are then maintained under agitation in a water bath heated to 80° C.

Protocol for preparation of the viscosity-increasing solution (ie BASE or BF): the sodium hydroxide and water are mixed by means of a magnetic bar for a period of 5 minutes, in such a manner as to obtain a 10% sodium hydroxide solution.

### Microfluidic Method

The IF solution heated to 80° C. is introduced into a first syringe (ie IF syringe) connected to a heater that enables the solution to be kept warm.

The OF solution is also heated to 80° C. and introduced into a second syringe (ie OF syringe) connected to a heater that enables the solution to be kept warm.

In order to reduce heat loss, the microfluidic device is installed directly at the outlet of the IF and OF syringes.

The base, maintained at ambient temperature, is introduced into a third syringe (i.e. BF syringe), which is also connected to the microfluidic device but after the outlet of the microfluidic nozzles.

The IF, OF and BF are subsequently injected into a microfluidic device with flow rates that are adapted so as to ensure the presence of these solutions in the final composition, in accordance with the flow rates and percentages described here below.

| Phase | Flow rates (in ml/hour/nozzle) |
|---|---|
| OF | 75 |
| IF | 8.07 |
| BF | 8.33 |
| Total | 91.4 |

Over the course of the manufacturing process, the diameter of the dispersed fatty phase droplets was observed, thereby providing information on the stability (or robustness) of this manufacturing process and, in particular, on the appropriateness of the selection of raw materials. In fact, in a microfluidic manufacturing method, it is known that a change in the size of the droplets of a macroscopic emulsion, or indeed even the non-formation of droplets, can be symptomatic of problems relating to compatibility/miscibility between two raw materials, surface tension between the two phases, or viscosity, which may result in particular in an uncontrolled transition from a hydrodynamic mode referred to as “dripping” to a mode referred to as “jetting” (formation of a liquid jet at the outlet of the microfluidic device), and/or clogging of the microfluidic nozzle.

The coefficient of variation Cv of the average diameters D̅ (1000 microns) of the dispersed phase droplets as measured over a manufacturing time period of 5 hours (in accordance with the method described in the description) for the compositions 2A and 2B is less than 3% (measured value 2.04%). This difference in the diameter of the droplets is therefore negligible.

In an unexpected fashion, it is clearly apparent from this example that the modifications/substitutions effected in order to transition from the comparative composition 2A to the composition 2B according to the invention, namely (i) dispensing with using amodimethicone, and therefore a membrane (the role of which is to ensure the mechanical resistance of the droplets, and therefore the kinetic stability of the emulsion), and (ii) satisfying the requirement for a percentage of ingredients of natural origin that is greater than 95%, are not detrimental to the implementation, or even to the robustness, of the microfluidic manufacturing method.

The percentage of ingredients of natural origin obtained for composition 2B even serves to enable the claim to using the classification of “natural product” within the meaning of standard ISO 16128.

The tests and results in terms of kinetic stability and sensorial properties are described in Example 4 here below.

### Example 3: Macroscopic Direct Emulsion for Skin Care

A composition 3 was prepared in the form of a macroscopic direct emulsion by using a microfluidic method as described in the patent document WO2015055748. The formulas for the continuous aqueous phase and the dispersed fatty phase are described in the Table 3 here below.

TABLE 3
FINAL COMPOSITION 3

| | | % w/w | % w/w | Natural | % |
|---|---|---|---|---|---|
| Name | INCI name | Phases | Final | index* | Natural* |
| AQUEOUS GEL | Sub total | 100.00 | 92 | | |
| PHASE (OF) | | | | | |
| REVERSE OSMOSIS | AQUA | 81.62 | 75.08 | 1 | 75.0886 |
| WATER | | | | | |
| GLYCERINE Codex | Glycerin & Aqua | 5.43 | 5.00 | 1 | 5 |
| GENENCARE OSMS BA | Betaine | 4.425 | 4.071 | 1 | 4.071 |
| ZEMEA SELECT | Propanediol & | 4.35 | 4.00 | 1 | 4 |
| PROPANEDIOL | Aqua | | | | |
| A-LEEN 5/PENTIOL | PENTYLENE | 2.17 | 2.00 | 1 | 2 |
| GREEN+ | GLYCOL, AQUA | | | | |
| HEXIOL | 1,2-Hexanediol & | 1.08 | 1.00 | 0 | 0 |
| | Aqua | | | | |
| CARBOPOL ULTREZ | CARBOMER | 0.39 | 0.36 | 0 | 0 |
| 10 POLYMER | | | | | |
| CITRIC ACID | Citric Acid | 0.17 | 0.1564 | 0 | 0 |
| ANHYDROUS PRS | | | | | |
| CODEX | | | | | |
| NATRLQUEST E30 | Aqua & Trisodium | 0.16 | 0.15 | 0.63 | 0.0945 |
| | Ethylenediamine | | | | |
| | Disuccinate | | | | |
| ALCASEALAN | Alcaligenes | 0.02 | 0.0175 | 1 | 0.0175 |
| | Polysaccharides | | | | |
| SODIUM | HYDROXIDE | 0.17 | 0.1565 | 0 | 0 |
| HYDROXIDE | SODIUM | | | | |
| PELLETS PRS | | | | | |
| CODEX | | | | | |
| OILY PHASE (IF) | Sub total | 100.00 | 8 | | |
| Labrafac CC | Caprylic/Capric | 66.62 | 5.33 | 1 | 5.33 |
| | triglyceride | | | | |
| EMC30 | Castor Oil/IPDI | 33.33 | 2.6667 | 0.974 | 2.597 |
| | Copolymer (and) | | | | |
| | Caprylic/Capric | | | | |
| | Triglyceride | | | | |

| | | |
|---|---|---|
| TOTAL | 100.0000 | 98.19 |

*according to the standard ISO 16128.
** EMC30: premix of Estogel M (INCI: Castor Oil/IPDI Copolymer (and) Caprylic/Capric Triglyceride) in Caprylic/Capric Triglyceride oil in a 30/70 ratio; in this instance, concentration of Estogel M = 10% relative to the weight of the phase that contains the latter.

Protocol for OF and IF preparation: the preparation protocols for preparing OF and IF fall within the scope of general knowledge of the person skilled in the art. The solutions obtained are then maintained under agitation in a water bath heated to 80° C.

Protocol for preparation of the viscosity-increasing solution (ie BASE or BF): the sodium hydroxide and water are mixed by means of a magnetic bar for a period of 5 minutes, in such a manner as to obtain a 10% sodium hydroxide solution.

Microfluidic Method: identical to that described in Example 2.

The IF, OF and BF are injected into the microfluidic device as described in the document WO2015055748 with flow rates that are adapted so as to ensure the presence of these solutions in the final composition, in accordance with the flow rates and percentages described here below.

| Phase | Flow rates (in ml/hour/nozzle) |
|---|---|
| OF | 75 |
| IF | 7.96 |
| BF | 8.33 |
| Total | 91.3 |

The coefficient of variation Cv of the average diameters D̅ (968 microns) of the dispersed phase droplets as measured over a manufacturing time period of 5 hours in accordance with the method described in the description) is less than 3% (measured value 1.92%). This difference in the diameter of the droplets is therefore negligible.

In an unexpected fashion, it is clearly apparent from this example that the choices of raw materials made in order to achieve a percentage of ingredients of natural origin that is greater than 95% are not detrimental to the implementation, or even to the robustness, of the microfluidic manufacturing method.

The percentage of ingredients of natural origin obtained for composition 2B even serves to enable the claim to using the classification of “natural product” within the meaning of standard ISO 16128.

The tests and results in terms of kinetic stability and sensorial properties are described in Example 4 here below.

### Example 4: Stability and Sensorial Properties

### Stability Tests

The Compositions 2A, 2B, and 3 in the examples here above are each packaged in three 30 ml polypropylene (PP) receptacles that are half-filled.

After 1 day at ambient temperature, each assay is subjected to one of the following three transport tests (one receptacle per test), namely:
roller test (i.e. horizontal circular movement): reference Wheaton, for a period of 1 hour;
vibrating table (i.e. vertical circular movement): reference Heidolph Unimax 1010, for a period of 1 hour; and
3D mixer (i.e. random movement): for a period of 6 minutes.

On completion of these stability tests, the following are assessed: (i) the integrity of the droplets, in particular the fragmentation thereof; and (ii) the turbidity of the continuous phase for the direct emulsions, generally linked to a transfer of the fatty phase into the aqueous phase.

### Scoring Criteria

| Scoring | | | | |
|---|---|---|---|---|
| Criteria | 0 | 1 | 2 | 3 |
| Fragmentation | Absence of | Slight | Moderate | Significant |
| of bubbles | fragmen- | fragmen- | fragmen- | fragmen- |
| | tation | tation | tation | tation |
| Turbidity of | Transparent | Slightly | Moderately | Cloudy gel |
| the gel | gel | cloudy gel | cloudy gel | |

Scores between 0 and 1 shall be considered to be a satisfactory result.

### Results

| Composition | Ex. 2A | Ex. 2B | Ex. 3 |
|---|---|---|---|
| FRAGMENTATION OF | 1 | 1 | 1 |
| BUBBLES | | | |
| TURBIDITY OF THE GEL | 1 | 1 | 1 |

The compositions tested all presented satisfactory stability results.

### Sensory Tests

Visual and sensory tests were carried out on a cohort of 24 women aged between 22 and 45 years, with each woman being subjected to a blind test of the compositions 2A, 2B, 3 and 4.

The composition 4 differs from Example 2B in that it has a Rheopearl KL2 content of 20% by weight relative to the total weight of the dispersed phase, subject to adjustment of the content of CETIOL C5 C (INCI: Coco-Caprylate/Caprate).

The criteria evaluated were as follows: (i) aggregation of the droplets of dispersed phase with each other; (ii) ease (or comfort) of application, and in particular, ease as regards flattening and spreading the droplets of dispersed phase; and (iii) hydration.

### Scoring Criteria

| SCORING | | | | |
|---|---|---|---|---|
| CRITERIA | 0 | 1 | 2 | 3 |
| AGGREGATION | Absence of | Slight | Moderate | High aggregation |
| | aggregation | aggregation | aggregation | |
| EASE OF | Very satisfactory | Satisfactory | Moderately | Unsatisfactory |
| APPLICATION | application. | application. | satisfactory | application. |
| | The droplets are not | The droplets | application. | The droplets are felt |
| | (or only very | are very slightly | The droplets | and upon being |
| | slightly) felt and | felt and mixing | are felt and | flattened on the skin, |
| | mixing between the | between the | upon being | they generate clumps |
| | aqueous and fatty | aqueous and | flattened on the | that are difficult to |
| | phases is easy | fatty phases is | skin, they | break up by |
| | effected. | easily effected. | generate | spreading over the |
| | | | clumps. | skin. The droplets |
| | | | Application of | have an excessively |
| | | | the dispersion | high degree of |
| | | | remains | hardness (firmness). |
| | | | possible. | |
| HYDRATION | Very satisfactory | Satisfactory | Moderately | Unsatisfactory |
| | hydration | hydration | satisfactory | hydration |
| | | | hydration | |

Scores between 0 and 1 shall be considered to be a very satisfactory result.

A score of 2 shall be considered to be a satisfactory result.

### Results

| | Ex. 2A | | | |
|---|---|---|---|---|
| Composition | (comparative) | Ex. 2B | Ex. 3 | Ex. 4 |
| AGGREGATION | 2 | 2 | 1 | 2 |
| EASE OF | 1 | 0 | 1 | 2 |
| APPLICATION | | | | |
| HYDRATION | 1 | 0 | 0 | 0 |

*NR:Not provided.

### Conclusion

It appears that a composition according to the invention, that is to say in the form of a macroscopic emulsion free of amodimethicone (and thus also membrane free) provided with a percentage of ingredients of natural origin that is greater than or equal to 95% (according to the standard ISO 16128), in an unexpected manner continues to retain satisfactory or even enhanced properties in terms of kinetic stability, sensory properties (particularly in terms of comfort and ease of application) and hydration, such being the case despite the presence of droplets of dispersed phase of macroscopic size and/or high content levels of dispersed phase.

Finally, by comparison of Examples 2B and 4, it appears that a composition according to the invention has particularly satisfactory properties in terms of ease of application when the content of the one or more lipophilic gelling agent(s) is less than 20% by weight relative to the total weight of the fatty phase that contains the latter.

1. A composition which is stable in the form of an emulsion comprising at least one fatty phase and at least one aqueous phase which are immiscible with each other at ambient temperature and atmospheric pressure and in which the dispersed phase is in the form of droplets, wherein:
the droplets of dispersed phase that have a diameter greater than or equal to 250 μm represent a volume greater than or equal to 60%, and/or at least 60% of the droplets have an average diameter greater than or equal to 250 μm;
the composition comprises from 1% to 60% by weight of dispersed phase relative to the total weight of the composition;
the composition comprises a percentage of ingredients of natural origin that is greater than or equal to 95%; and
the composition is free of amodimethicone.

2. A composition according to claim 1, in which the emulsion is in the form of a single emulsion.

3. A composition according to claim 1, wherein the droplets of dispersed phase do not comprise a shell.

4. A composition according to claim 1, wherein the fatty phase comprises a percentage of ingredients of natural origin that is greater than or equal to 95%.

5. A composition according to claim 1, wherein the droplets of dispersed phase are monodisperse.

6. A composition according to claim 1, further comprising at least one hydrophilic gelling agent.

7. A composition according to claim 1, further comprising at least one lipophilic gelling agent.

8. A composition according to claim 1, in which the fatty phase has a melting point of between 50° C. and 100° C., and, at ambient temperature and atmospheric pressure, satisfies the following physicochemical criteria:
a hardness (x) of between 2 and 14 N; and
a tackiness (y) greater than or equal to −2 N; and/or
optionally, a cohesiveness (z) less than or equal to 40.

9. A composition according to claim 1, wherein the composition does not comprise any surfactant.

10. A method for preparing a composition according to claim 1, that includes at least the following steps:
a) providing at least one oily fluid FH in liquid form and at least one aqueous fluid FA in liquid form, these being immiscible with each other;
b) injecting the aqueous fluid FA into the oily fluid FH under agitation, or vice versa, in order to obtain an emulsion comprising droplets of dispersed phase, constituted of the oily fluid FH or the aqueous fluid FA, in a continuous phase constituted of the other of the aqueous fluid FA or the oily fluid FH;
c) optionally, cooling the composition obtained in the step (b) to ambient temperature or to a temperature lower than ambient temperature, and
d) recovering the composition in emulsion form.

11. A method for preparing a composition according to claim 10, that includes at least the following steps:
a) providing at least one oily fluid FH in liquid form and at least one aqueous fluid FA in liquid form, which are immiscible with each other;
b) injecting the aqueous fluid FA and the oily fluid FH into a microfluidic device that is capable of forming droplets of dispersed phase, constituted of the oily fluid FH or the aqueous fluid FA, in which the continuous phase, constituted of the other of the aqueous fluid FA or the oily fluid FH, circulates continuously;
c) optionally, cooling the composition obtained in the step (b) to ambient temperature or to a temperature lower than ambient temperature; and
d) continuously recovering the composition in emulsion form.

12. A non-therapeutic process for the cosmetic treatment of a keratinous material, which includes a step of applying to the said keratinous material at least one composition according to claim 1.

13. The composition according to claim 1, wherein the droplets of dispersed phase do not comprise a shell formed by a layer of coacervate interposed between the dispersed fatty phase and the continuous aqueous phase.
